(19) United States
(12) Patent Application Publication
(10) Pub. No.: US 2025/0018408 A1
(43) Pub. Date: Jan. 16, 2025

(54) MODULAR SHOWER HEAD WITH REMOVABLE SPOUT ASSEMBLY AND AROMA PUCK ATTACHMENT

(71) Applicant: Hyku Home Inc., Austin, TX (US)

(72) Inventors: Justin Seidenfeld, Brooklyn, NY (US); Lucas Lerman Lappe, Brooklyn, NY (US)

(73) Assignee: Hyku Home Inc., Austin, TX (US)

(21) Appl. No.: 18/771,694

(22) Filed: Jul. 12, 2024

Related U.S. Application Data
(60) Provisional application No. 63/526,391, filed on Jul. 12, 2023.

(51) Int. Cl. B05B 7/24

(52) U.S. Cl. CPC B05B 7/24

(57) ABSTRACT

The present disclosure relates to a shower head which may comprise a shroud assembly and a spout assembly. The shroud assembly may include an inner shroud with alignment tracks and a female quick release. The spout assembly may include a male quick release, nubs, a filter assembly housing, and a faceplate with openings and a magnet for interfacing with a magnetic plate. The male and female quick releases may allow for removable attachment of the spout assembly to the shroud assembly. The alignment tracks and nubs may temporarily prevent full detachment of the spout assembly from the shroud assembly. Additionally, the shower head may include an aroma puck that is magnetically attached to the faceplate via a magnetic plate.

### FIELD OF THE DISCLOSURE

The present disclosure is directed to a shower head apparatus. More specifically, the present disclosure is directed to a shower head apparatus with a replaceable filter and aroma puck.

### INTRODUCTION

Various shower head designs have been developed to provide users with a comfortable and enjoyable showering experience. Traditional shower heads typically consist of a fixed nozzle or a handheld device that is connected to a water supply. While these designs are functional, they often lack versatility and customization options.

More recently, shower heads with detachable components have been introduced to the market. These designs allow users to easily remove and replace certain parts of the shower head. However, these detachable shower heads often require complex mechanisms or tools for attachment and detachment, making them less user-friendly and convenient.

Additionally, there has been a growing interest in incorporating aromatherapy into showering experiences. Aromatherapy can provide relaxation and therapeutic benefits through the use of scented oils or pucks. However, existing shower heads with aromatherapy features typically involve separate compartments or attachments for holding the aroma source, which can be cumbersome and may not provide a secure attachment.

The present disclosure aims to address these limitations by providing a shower head that incorporates a quick release mechanism for easy attachment and detachment of components, as well as a magnetic attachment system for securely holding an aroma puck.

### SUMMARY

Aspects of the present disclosure relate to a shower head, including: a shroud assembly including an inner shroud and female quick release, wherein the inner shroud includes one or more alignment tracks; a spout assembly including a male quick release, one or more nubs, a filter assembly housing, and a faceplate, wherein the faceplate includes one or more openings, and a magnet configured to interface with a magnetic plate, wherein the male quick release and female quick release are configured to allow the spout assembly to removably attach to the shroud assembly, and wherein the one or more alignment tracks are configured to interface with the one or more nubs to temporarily prevent full detachment of the spout assembly from the shroud assembly.

Aspects of the present disclosure relate to a shower head, wherein the filter assembly housing includes a filter assembly disposed within an interior cavity defined by the filter assembly housing.

Aspects of the present disclosure relate to a shower head, further including an aroma puck, wherein the aroma puck is magnetically attached to the faceplate via a magnetic plate.

Aspects of the present disclosure relate to a shower head, wherein the female quick release includes one or more snaps configured to interface with one or more ridges disposed on the male quick release.

Aspects of the present disclosure relate to a shower head, wherein the inner shroud further includes at least one shelf member disposed within the one or more alignment tracks.

Aspects of the present disclosure relate to a shower head, further including a ball joint configured to be coupled to the shroud assembly via a ball joint interface.

Aspects of the present disclosure relate to a shower head, wherein the ball joint interface defines an inner circumference, and wherein the ball joint interface includes a gasket disposed around the inner circumference.

Aspects of the present disclosure relate to a shower head, wherein the ball joint includes a protrusion extending radially outward from an outside surface of the ball joint.

Aspects of the present disclosure relate to a shower head, further including an adjustor operatively connected to the faceplate.

Aspects of the present disclosure relate to a shower head, wherein the adjustor is configured to selectively allow a passage of liquid through one or more openings located on the faceplate.

Aspects of the present disclosure relate to a shower head, including: a shroud assembly including an inner shroud and female quick release, wherein the inner shroud includes one or more alignment tracks; a spout assembly including a male quick release and one or more nubs, wherein the one or more alignment tracks are configured to interface with the one or more nubs to temporarily prevent full detachment of the spout assembly from the shroud assembly.

Aspects of the present disclosure relate to a shower head, further including a filter assembly housing, wherein the filter assembly housing includes a filter assembly disposed within an interior cavity defined by the filter assembly housing.

Aspects of the present disclosure relate to a shower head, wherein the filter assembly housing includes a plurality of passages.

Aspects of the present disclosure relate to a shower head, wherein the female quick release includes one or more snaps configured to interface with one or more ridges disposed on the male quick release.

Aspects of the present disclosure relate to a shower head, wherein the inner shroud further includes at least one shelf member disposed within the one or more alignment tracks.

Aspects of the present disclosure relate to a shower head, further including a ball joint configured to be coupled to the shroud assembly via a ball joint interface.

Aspects of the present disclosure relate to a shower head, wherein the ball joint interface defines an inner circumference, and wherein the ball joint interface includes a gasket disposed around the inner circumference.

Aspects of the present disclosure relate to a shower head, wherein the ball joint includes a protrusion extending radially outward from an outside surface of the ball joint.

Aspects of the present disclosure relate to a shower head, including: a shroud assembly including an inner shroud and female quick release; a spout assembly including a male quick release, wherein the male quick release and female quick release are configured to allow the spout assembly to removably attach to the shroud assembly via one or more snaps disposed on the female quick release and configured to interface with one or more ridges disposed on the male quick release.

Aspects of the present disclosure relate to a shower head, further including a ball joint configured to be coupled to the shroud assembly via a ball joint interface.

Additional aspects related to this disclosure are set forth, in part, in the description which follows, and, in part, will be obvious from the description, or may be learned by practice of this disclosure.

It is to be understood that both the forgoing and the following descriptions are exemplary and explanatory only and are not intended to limit the claimed disclosure or application thereof in any manner whatsoever.

### BRIEF DESCRIPTION OF THE DRAWINGS

FIG. 1A illustrates a shower head according to various aspects of the present disclosure.

FIG. 1B shows an exploded view of a shower head according to aspects of the present disclosure.

FIG. 2 shows an exploded view of a spout assembly according to aspects of the present disclosure.

FIG. 3 shows a cross-sectional view of the shower head according to aspects of the present disclosure.

FIG. 4 shows a shower head connected to a supply pipe according to aspects of the present disclosure.

FIGS. 5-6 illustrate the interface between the shroud assembly and spout assembly according to aspects of the present disclosure.

FIG. 7 illustrates a relationship between a shroud assembly and spout assembly according to aspects of the present disclosure.

FIG. 8 illustrates a faceplate according to various aspects of the present disclosure.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized, and that structural changes and/or substitutions of various elements may be made without departing from the scope and spirit of this disclosure. The following detailed description is, therefore, not to be construed in a limited sense.

It is noted that the description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

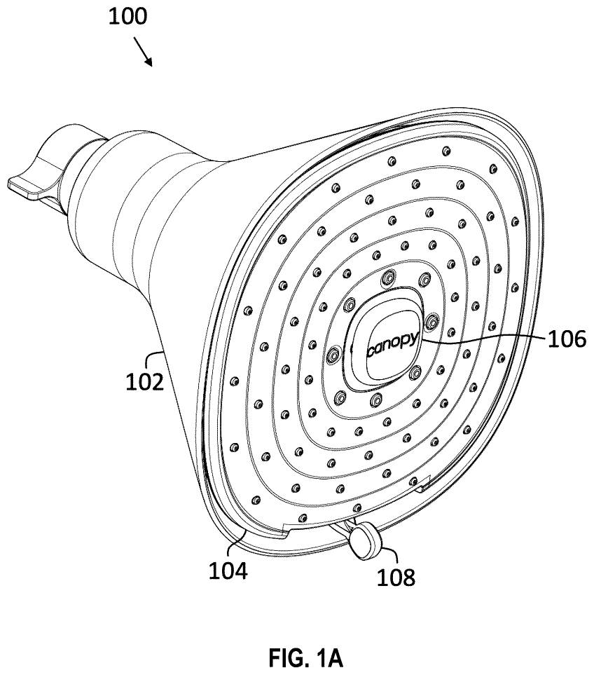

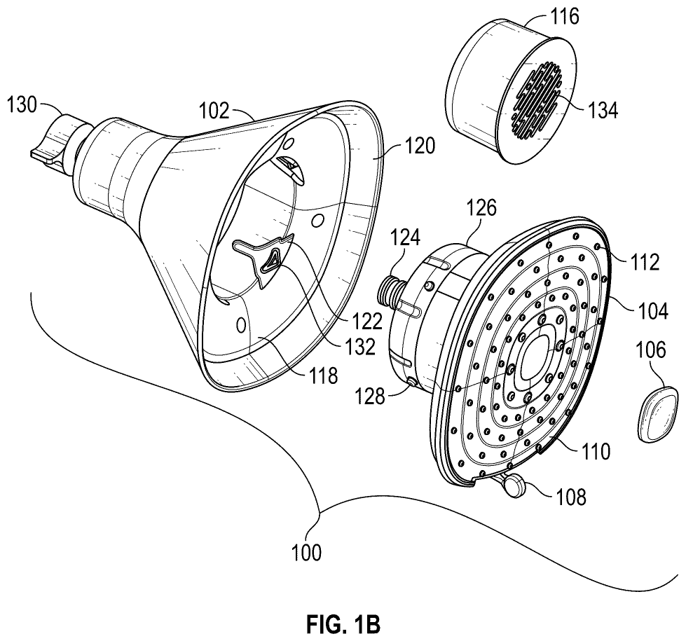

FIG. 1A illustrates a shower head 100 according to various aspects of the present disclosure. The shower head 100 may include a number of components including a shroud assembly 102, a spout assembly 104, a filter assembly 116, and/or an aroma puck 106.

Turning to FIG. 1B, the shower head 100 may be easily disassembled into its constituent parts, the shroud assembly 102, spout assembly 104, filter assembly 116, and aroma puck 106 as shown. Thus, the modular nature of the shower head 100 makes the shower head 100 easy to use and disassemble, which is particularly useful for replacing interior components, such as filters enclosed in the filter assembly 116.

The filter assembly 116 may include plurality of passages 134 on both sides of the filter assembly 116 to allow for fluid communication from the male quick release 124 through to the faceplate 110. The filter assembly 116 may be separated into two parts exposing an interior cavity for placing a filter (not shown). Such filter may be configured to remove particulates from a supply of liquid. In an embodiment, the filter is configured to add minerals to the liquid, making it ideal for washing the skin/hair of a user.

As discussed in further detail below, the shroud assembly 102 may interface with the spout assembly 104 to securely attach the spout assembly 104 to the shroud assembly 102 and inner shroud 118. The filter assembly 116, which may contain a filter within it, may be secured inside the spout assembly 104 prior to assembly of the shower head 100. Moreover, the shower head 100 may include an aroma puck 106 which may be selectively attached to the spout assembly 104.

The shroud assembly 102 may be constructed from a resilient water-resistant material such as plastic. However, other suitable materials, such as metal, may be used. In an embodiment, the shroud assembly 102 forms a conical shape where sides of the shroud assembly 102 are tapered. However, other form factors are contemplated including cylindrical, cuboid, or other suitable form factor as known by those of skill in the art.

The shroud assembly 102 may include an inner shroud 118 disposed within an interior space defined by an internal surface 120 of the 102. The inner shroud 118 may be configured to secure the spout assembly 104 within the shroud assembly 102 and/or prevent the spout assembly 104 from unintentionally falling out of the shroud assembly 102 via one or more alignment tracks 122 (discussed in greater detail below).

The spout assembly 104 may include a male quick release 124, filter assembly housing 126, one or more nubs 128, adjustor 108, faceplate 110, and/or openings 112. The spout assembly 104 may be configured to be easily detachable from the shroud assembly 102 for purposes of accessing the interior space of the filter assembly housing 126. In an embodiment, the male quick release 124 is configured to interface with the female quick release 302 to create a watertight connection between the spout assembly 104 and the shroud assembly 102, thus enabling a flow of liquid from an entrance of the ball joint 130 through the openings 112 of the faceplate 110.

In an embodiment, the one or more nubs 128 interface with the one or more alignment tracks 122 to guide the spout assembly 104 into the shroud assembly 102 and prevent the spout assembly 104 from unintentionally falling out. As a non-limiting example, the one or more alignment tracks 122 includes at least one shelf member 132 configured to catch the one or more nubs 128. In such an embodiment, slight rotation of the spout assembly 104 is required to move the one or more nubs 128 around the at least one shelf member 132 and completely detach the spout assembly 104 from the shroud assembly 102.

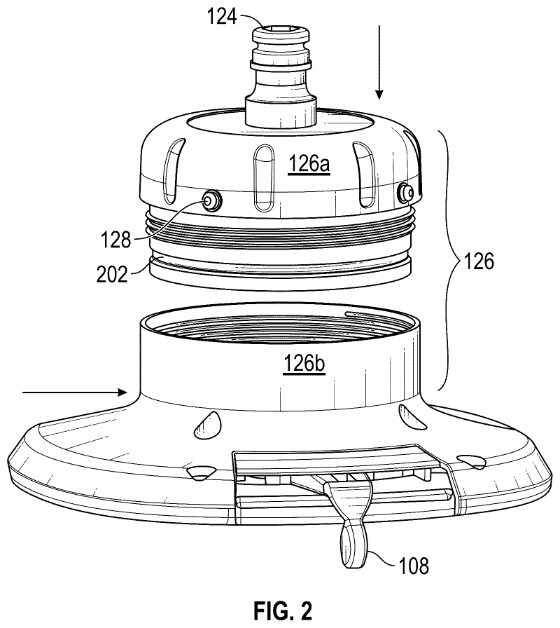

The filter assembly 116 may contain a filter (not shown) to treat liquid passing through the shower head 100. To allow for fluid transmission through the filter assembly 116, the filter assembly 116 may include a plurality of passages 134. As shown in FIG. 2, the filter assembly housing 126 may include a first segment 126a and a second segment 126b, which may be separated to reveal an internal space defined by the filter assembly housing 126 and configured to house the filter assembly 116.

First segment 126a may include a threaded outer circumference which may interface with a threaded inner circumference of the second segment 126b to enable a secure coupling of the first segment 126a to the second segment 126b. Other coupling mechanisms may be used, such as an interference fit, or any other suitable fit known to those of skill in the art. The first segment 126a may include a gasket 202 disposed around a circumference of the first segment 126a. The gasket 202 may provide a watertight fit to ensure that liquid travels along the intended path through the shower head 100. The gasket 202 may, additionally or alternatively, be disposed along a circumference of the second segment 126b.

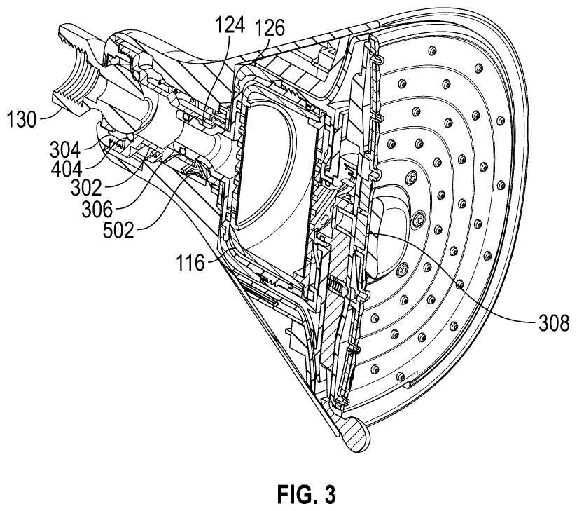

FIG. 3 shows a cross-sectional view of the shower head 100 according to aspects of the present disclosure. In an embodiment, the shroud assembly 102 includes a female quick release 302, the inner shroud 118, and/or a ball joint interface 304.

The spout assembly 104 may include a magnet 308 abutting the faceplate 110 for purposes of securing a magnet 308 as discussed further below.

The spout assembly 104 may be configured to interface with an aroma puck 106 via the magnet 308. In an embodiment, the aroma puck 106 may include a diatomite puck and magnetic plate. The faceplate 110 may include the magnet 308 configured to interface with a corresponding magnetic plate within the aroma puck 106 to attach the aroma puck 106 to the faceplate 110. In an embodiment the magnet 308 is located at an approximate center of the faceplate 110. However, the magnet 308 may be positioned at any suitable location of the faceplate 110.

The male quick release 124 and female quick release 302 may interface to couple the spout assembly 104 to the shroud assembly 102. When inserted into the female quick release 302, a gasket 306 may ensure that a watertight seal is achieved. The gasket 306 may extend around a circumference of the male quick release 124.

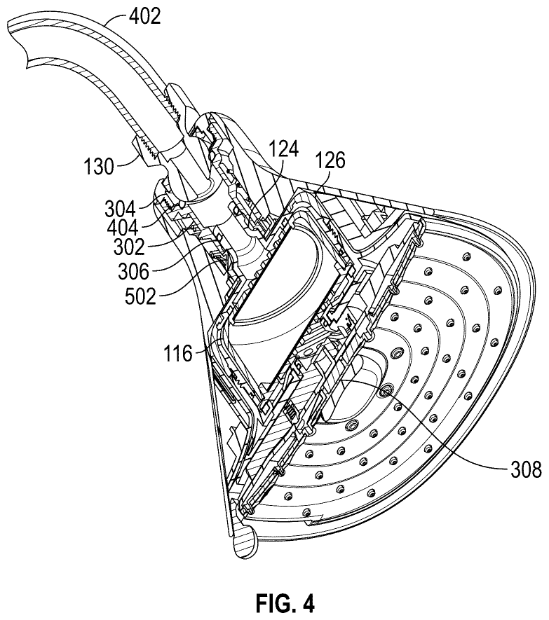

FIG. 4 shows the shower head 100 connected to a supply pipe 402 according to aspects of the present disclosure. The at least one ball joint 130 allows for a secure connection to be made to the supply pipe 402 while also enabling mobility of the shower head 100 about the ball joint 130. The ball joint 130 may include a threaded flange allowing a user to install the shower head 100 onto a supply pipe 402 having complimentary threading. The ball joint 130 may include a protrusion extending radially outward from an outside surface of the ball joint 130 as shown in FIG. 1B. Such protrusion allows a user to easily tighten the ball joint 130 to the supply pipe 402 without the need for wrenches or additional tools.

The “ball” portion of the ball joint 130 may sit in a ball joint interface 304. In an embodiment, the ball joint interface 304 includes a gasket 404 extending around an inner circumference of the ball joint interface 304 to provide a watertight seal against the ball joint 130, while permitting movement of the shower head 100 about the ball joint 130.

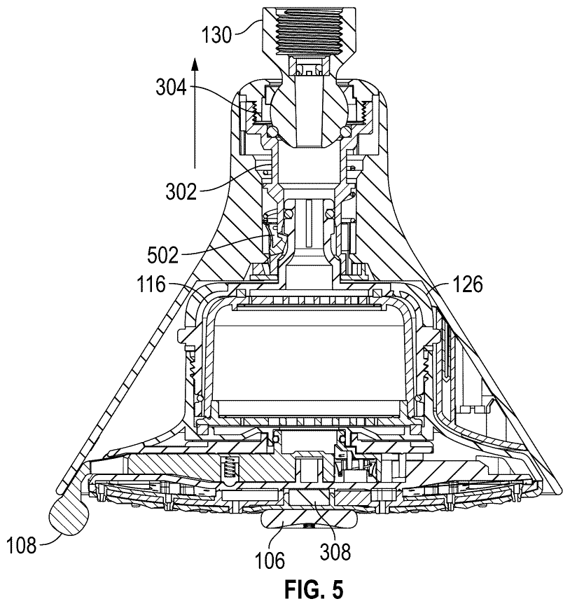

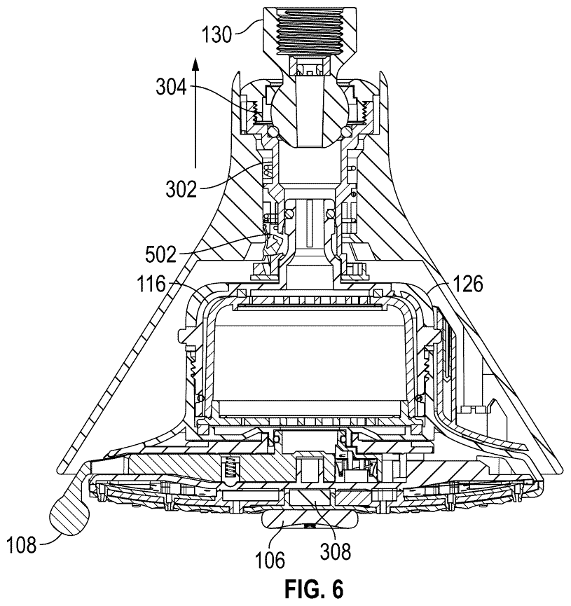

FIGS. 5-6 illustrate the interface between the shroud assembly 102 and spout assembly 104. In an embodiment, the female quick release 302 includes one or more snaps 502 which allow a user to selectively detach the spout assembly 104 from the shroud assembly 102. In an embodiment, the female quick release 302 includes three snaps 502. However, any suitable quantity of snaps 502 may be used.

As shown in FIG. 5, the spout assembly 104 may be fitted into the shroud assembly 102 in a home state. When in the home state, the snaps 502 may prevent the male quick release 124 from sliding out of the female quick release 302 as illustrated in FIG. 5. In such an embodiment, the male quick release 124 may include one or more ridges which directly interface with the one or more snaps 502.

The snaps 502 may be spring-loaded such that, in a home state, the snaps 502 are disengaged from the male quick release 124 (discussed further below). The shroud assembly 102 may further include a ridge that forces the snaps 502 to engage with the male quick release 124 when the spout assembly 104 is in its home state.

Turning to FIG. 6, a user a push the shroud assembly 102 in an upward direction, which may displace the shroud assembly 102 upward relative to the spout assembly 104, female quick release 302, ball joint interface 304, and ball joint 130. Such displacement may then provide an area adjacent to each of the snaps 502 to allow each of the snaps 502 to disengage. Once disengaged, the male quick release 124 may be separated from the female quick release 302. Such functionality may permit a user to easily separate the spout assembly 104 and replace the filter assembly 116. In an embodiment, a spring is utilized around the female quick release 302 to keep the spout assembly 104 in its home state absent an external force applied by the user.

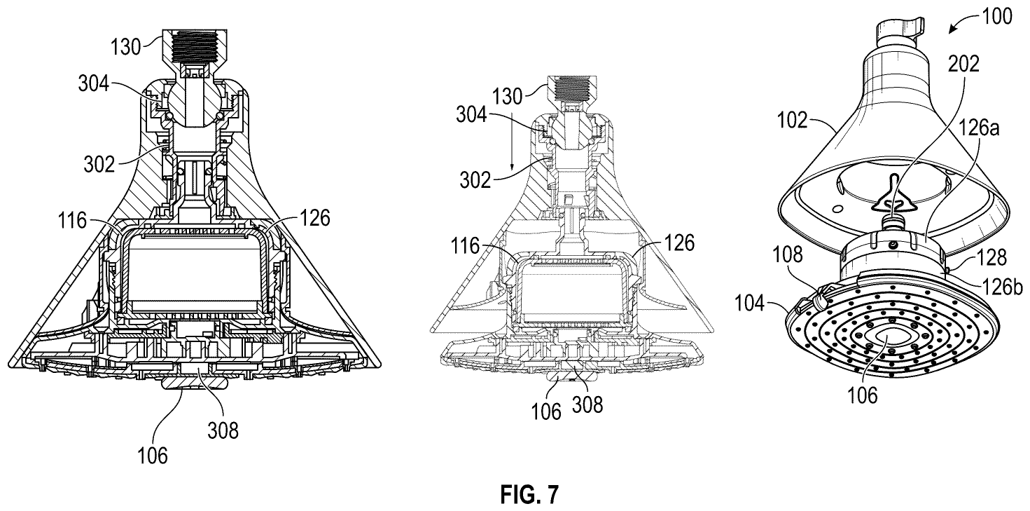

To prevent the spout assembly 104 from unintentionally falling out of the shroud assembly 102, the spout assembly 104 may include one or more nubs 128 as shown in FIG. 7. In an embodiment, the one or more nubs 128 are configured to interface with one or more alignment tracks 122. Once the male quick release 124 has been released, the entire spout assembly 104 may move downward along the one or more alignment tracks 122 until the one or more nubs 128 are caught by at least one shelf member 132 located on the inner shroud 118. The user may then rotate the loose spout assembly 104 either clockwise or counter-clockwise to free the one or more nubs 128 from the one or more alignment tracks 122 and fully remove the spout assembly 104.

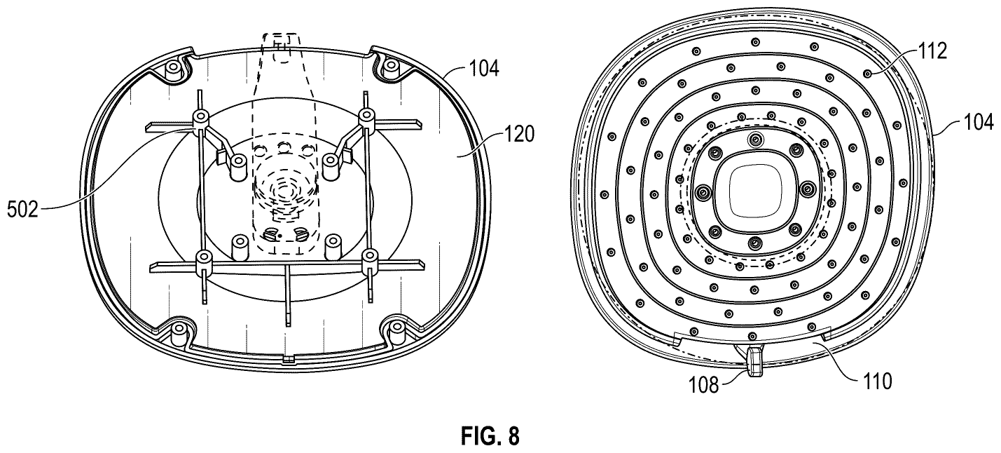

FIG. 8 illustrates the faceplate 110 according to various aspects of the present disclosure. The adjustor 108 may be interacted with to adjust the spray pattern of the shower head 100 when in use. For example, the adjustor 108 may by positioned into one of several positions. In an embodiment, there are three positions. Each position may correspond to one or more specific openings 112 of the faceplate 110. There may be several configurations of openings 112 corresponding to each of the several positions. As a non-limiting example, the configurations may be any one of a high-pressure, low-pressure, or pause configuration. The high-pressure configuration, indicated be the left arrow, may correspond to openings shown in the inner circle of the faceplate 110.

The low-pressure configuration, indicated by the middle arrow, may correspond to the openings 112 shown between the inner and outer circles of the faceplate 110. The pause configuration, indicated by the right arrow, may correspond to the openings 112 shown between the inner and outer circles of the faceplate 110 shown above. In an embodiment, the channel through which water flows may be restricted in the pause configuration so as to minimize the flow of water.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A shower head, comprising:
a shroud assembly including an inner shroud and female quick release,
wherein the inner shroud includes one or more alignment tracks;
a spout assembly including a male quick release, one or more nubs, a filter assembly housing, and a faceplate,
wherein the faceplate includes one or more openings, and a magnet configured to interface with a magnetic plate,
wherein the male quick release and female quick release are configured to allow the spout assembly to removably attach to the shroud assembly, and
wherein the one or more alignment tracks are configured to interface with the one or more nubs to temporarily prevent full detachment of the spout assembly from the shroud assembly.

2. The shower head of claim 1, wherein the filter assembly housing includes a filter assembly disposed within an interior cavity defined by the filter assembly housing.

3. The shower head of claim 1, further including an aroma puck, wherein the aroma puck is magnetically attached to the faceplate via a magnetic plate.

4. The shower head of claim 1, wherein the female quick release includes one or more snaps configured to interface with one or more ridges disposed on the male quick release.

5. The shower head of claim 1, wherein the inner shroud further includes at least one shelf member disposed within the one or more alignment tracks.

6. The shower head of claim 1, further including a ball joint configured to be coupled to the shroud assembly via a ball joint interface.

7. The shower head of claim 6, wherein the ball joint interface defines an inner circumference, and wherein the ball joint interface includes a gasket disposed around the inner circumference.

8. The shower head of claim 6, wherein the ball joint includes a protrusion extending radially outward from an outside surface of the ball joint.

9. The shower head of claim 1, further including an adjustor operatively connected to the faceplate.

10. The shower head of claim 9, wherein the adjustor is configured to selectively allow a passage of liquid through one or more openings located on the faceplate.

11. A shower head, comprising:
a shroud assembly including an inner shroud and female quick release,
wherein the inner shroud includes one or more alignment tracks;
a spout assembly including a male quick release and one or more nubs,
wherein the one or more alignment tracks are configured to interface with the one or more nubs to temporarily prevent full detachment of the spout assembly from the shroud assembly.

12. The shower head of claim 11, further including a filter assembly housing, wherein the filter assembly housing includes a filter assembly disposed within an interior cavity defined by the filter assembly housing.

13. The shower head of claim 12, wherein the filter assembly housing includes a plurality of passages.

14. The shower head of claim 11, wherein the female quick release includes one or more snaps configured to interface with one or more ridges disposed on the male quick release.

15. The shower head of claim 11, wherein the inner shroud further includes at least one shelf member disposed within the one or more alignment tracks.

16. The shower head of claim 11, further including a ball joint configured to be coupled to the shroud assembly via a ball joint interface.

17. The shower head of claim 16, wherein the ball joint interface defines an inner circumference, and wherein the ball joint interface includes a gasket disposed around the inner circumference.

18. The shower head of claim 16, wherein the ball joint includes a protrusion extending radially outward from an outside surface of the ball joint.

19. A shower head, comprising:
a shroud assembly including an inner shroud and female quick release;
a spout assembly including a male quick release,
wherein the male quick release and female quick release are configured to allow the spout assembly to removably attach to the shroud assembly via one or more snaps disposed on the female quick release and configured to interface with one or more ridges disposed on the male quick release.

20. The shower head of claim 19. further including a ball joint configured to be coupled to the shroud assembly via a ball joint interface.
